(19) United States
(12) Patent Application Publication
(10) Pub. No.: US 2012/0010299 A1
(43) Pub. Date: Jan. 12, 2012

(54) Non-prescription medicine, vital fluid, and/or nutritional supplement

(76) Inventors: Brian Kaplan, Palm Beach Gardens, FL (US); Robert Terwilliger, Palm Beach Gardens, FL (US); Richard Haskins, Palm Beach Gardens, FL (US); Paul Schlie, Durham, NH (US)

(21) Appl. No.: 13/169,651

(22) Filed: Jun. 27, 2011

Related U.S. Application Data
(60) Provisional application No. 61/362,665, filed on Jul. 8, 2010.

(51) Int. Cl. A61K 31/09; A61P 11/10

(52) U.S. Cl. 514/718

(57) ABSTRACT

The present invention is directed toward a method of administering an oral solution containing one or more active ingredients, preferably formulations for non-prescription medicines, vital fluids, and/or nutritional supplements, optimally dosed based upon a patient's weight. The method includes providing an oral solution having an effective amount of one or more active ingredients; determining the weight of the individual, preferably to an individual less than 12 years old; and administrating an appropriate amount of the oral solution in a single dose dispensing unit in accordance with the weight determination of the individual, whereby the total amount of active ingredient administered corresponds to an effective amount of the active ingredient based on the individual's weight.

### REFERENCE TO RELATED APPLICATIONS

This application claims the benefit of U.S. Provisional Patent Application No. 61/362,665, filed on Jul. 8, 2010, entitled “Non-Prescription Medicine, Vital Fluid, and/or Nutritional Supplement”, the contents of which are incorporated herein by reference.

### FIELD OF THE INVENTION

The present invention relates to the field of medications, vital fluids, and/or nutritional supplements. More particularly, the present invention relates to a method of administering non-prescription medications, vital fluids, and/or nutritional supplements, and the non-prescription medications, vital fluids, and/or nutritional supplements formulated and labeled to be dispensed according to the method that delivers accurate and efficacious levels of active ingredients to children based upon the individual child's weight as opposed to age.

### BACKGROUND OF THE INVENTION

The pharmaceutical industry is a multi-billion dollar industry, representing large and small companies developing, producing and marketing numerous drugs for the treatment of a variety of medical conditions. Traditional routes followed by the pharmaceutical industry include the development of new chemical structures to cure specific diseases. Developing new chemical structures allows the pharmaceutical companies the opportunity to obtain limited market exclusivity by obtaining patent protection. While this route has generally been profitable, the patent life eventually expires. Increasing public pressure on these companies to lower the costs of drugs has also resulted in the development of alternative generic brands. Additionally, drug companies have been increasingly forced to compete for consumer dollars with the nutritional supplement industry. Although nutritional supplements can not be marketed as useful for curing disease, the industry has been very successful at providing products which can help a person live a healthier lifestyle. In an effort to maintain market share, drug companies have increasing been selling drugs which were previously available to consumers by prescription only as non-prescription alternatives. With increased use of non-prescription drugs, proper dosing and delivery is an important factor to consider when evaluating the effectiveness of such drugs.

The general concept of dosing of medicines based upon a patient's weight and/or other means of estimating their fluid volume is well know and commonly use by physicians. The dosing of non-prescription medicine, vital fluids, and/or nutritional supplements, particularly in children, is done based upon age groups, having only a very coarse correlation to weight or body surface area. Dosing based on age, thereby, results in a similarly coarse non-optimal therapeutic dosing of such medicine, vital fluids, and/or nutritional supplements. While earlier non-prescription medication dosing based on age may have been thought to provide the consumer with an easier option by requiring parents to know their child's age instead of their weight, age-based dosing can lead to inaccurate usage of the active ingredient, potentially exposing the children to harm.

Therefore, what is needed in the art is a method, and composition, for administration of non-prescription medicines, vital fluids, and/or nutritional supplements which provides a more optimally dosed administration scheme based upon a patient's weight rather than age.

### PRIOR ART

Although it has been recognized that weight-based dosing may provide advantages over age-based dosing, incorporating such a change to non-prescription medications has not been adopted by the FDA, which continues to publish monographs describing proper dosages for various active ingredients based on age. Current over-the-counter medications, according to FDA monographs, use the age of the individual as the standard for dispensing dosing units. In fact, the FDA requires that any medication whose dosing criteria are not based on age-related dosing regimens set forth in the current prescribing monograph be submitted for approval as a “new drug.” Several devices have been developed in an effort to aid the average consumer in dispensing non-prescription drugs in more accurate ways. For example, U.S. Pat. No. 6,276,533, describes a weight-specific elixir dosage calculation reference wherein a calculation aid is disclosed forming an integral part of a medication bottle or closure cap. The calculation aid, in essence, is a detailed weight-dose conversion chart, promoting the accurate dispensing of the weight-specific dosage of a given elixir to a child. The closure cap or bottle includes parallel rows of indicia for the weight of a child, calculated doses appropriate for the specific weight, and calculated quantity of elixir corresponding to the calculated dose. An aperture sleeve allows selected viewing of the indicia.

U.S. Pat. No. 6,581,773 is directed toward a weight-specific elixir dosage calculator, wherein a calculation aid is disclosed forming an integral part of a medication bottle. The calculation aid provides a reference chart for pediatric elixirs to assist in the accurate calculation of the weight-specific dosage for a child. The bottle includes parallel columns of patient's weights and rows of calculated quantity of elixir corresponding to the calculated dose. A movable indicator with an aperture allows selected viewing of the indicia.

U.S. Pat. No. 6,779,480 is directed to a dial indicator cap. More specifically, a rotatable dial is taught, with first and second windows mounted on top of a container closure cap; imprinted first set of information on top of the cap positioned to be sequentially viewable throughout its entire range through the first window upon indexed rotation of the dial; imprinted second set of information on top of the cap positioned to be sequentially viewable throughout its entire range through the second window when the first window is set to view a bit of the first set of information; and the second set of information is dependently related to the first set of information.

U.S. Pat. No. 6,904,867 is directed to a calculated dose medicine dispenser, particularly to a calculated medicine dose dispensing apparatus which includes a medicine bottle and a calculated dose medicine dispenser specific to the medicine bottle and a particular medicine. The medicine bottle has a bottle cap installed on its neck. The dose dispenser includes a cup in the shape of a truncated cone with a large diameter open end and an end wall closing the small diameter end. The end wall has a circular indent defined by an indent sidewall. The dose cup fits on the bottle cap with the large open end facing down and the bottle cap frictionally installed in the cup indent. A dial assembly is installed on the dose cup on the exterior of the end wall. The dial assembly calculates the proper dose of the medicine for the patient according to a patient parameter such as weight. The dial assembly includes a dial and a data disc. The data disc is installed on the dose cup end wall and carries at least two information sets that are circularly arranged, spaced apart information bits. Each information bit of one set is functionally related to an information bit of the other set, such as patient weight to medicine dose. The dial includes a dial plate installed over the data disc. The dial plate has one sight window corresponding to each information set. Rotation of the dial plate over the data disc displays functionally related information bits in the sight windows.

### SUMMARY OF THE INVENTION

The present invention a directed toward a method of administering an oral solution containing one or more active ingredients, preferably formulations for non-prescription medicines, vital fluids, and/or nutritional supplements optimally dosed based upon a patient's weight, or other means of estimating their fluid volume, with a minimum accuracy of 2 mL or less equivalent. The method includes providing an oral solution having an effective amount of one or more active ingredients, determining the weight of the individual, preferably an individual less than 12 years old; and administrating an appropriate amount of the oral solution in a dose dispensing unit in accordance with the weight determination of the individual. The total amount of active ingredient administered, therefore, corresponds to an effective amount of the active ingredient based on the individual's weight.

Accordingly, it is a primary objective of the instant invention to provide a method of administering an oral solution containing one or more active ingredients, and the oral solution formulated to dispense according to the dosing system, that incorporates an innovative, weight-based dosing scheme.

It is a further objective of the instant invention to provide a method of administering an oral solution containing one or more active ingredients in the form of an over-the-counter medication that incorporates an innovative, weight-based dosing scheme.

It is yet another objective of the instant invention to provide a method of administering an oral solution containing one or more active ingredients in the form of a vital fluid that incorporates an innovative, weight-based dosing scheme.

It is a further objective of the instant invention to provide a method of administering an oral solution containing one or more active ingredients in the form of a nutritional supplement that incorporates an innovative, weight-based dosing scheme.

It is yet another objective of the instant invention to provide a method of improving dosing in pediatric patients.

It is yet another objective of the instant invention to provide a method of improving dosing in pediatric patients which minimizes the risks associated with underdosing.

It is still yet another objective of the instant invention to provide to provide a method of improving dosing in pediatric patients which minimizes the risks associated with overdosing.

It is still yet another objective of the instant invention to provide a method of administering an oral solution containing one or more active ingredients in the form of an over-the-counter medication, a vital fluid, or a nutritional supplement based on the weight of the individual which is administered to children less than 12 years old.

It is yet another objective of the instant invention to provide a weight based method of administering an oral solution containing one or more active ingredients in the form of an over-the-counter medication, a vital fluid, or a nutritional supplement which minimizes the risks associated with under/overdosing of children less than 12 years old who are in the 90^{th} percentile for weight within their respective age group.

It is still yet another objective of the instant invention to provide a weight based method of administering an oral solution containing one or more active ingredients in the form of an over-the-counter medication, a vital fluid, or a nutritional supplement which minimizes the risks associated with under/overdosing of children less than 12 years old who are in the 10^{th} percentile for weight within their respective age group.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

The present invention is directed to a method of administering an oral solution, including over-the-counter (OTC) medications, vital fluids, and nutritional supplements, and the oral solution formulated to be administered according to the dosing scheme that incorporates an innovative, weight-based dosing system. The weight based dosing method in accordance with the present invention provides enhanced delivery of oral solutions containing one or more active ingredients. The dosing method, and solutions formulated to be administered according to the dosing method, is preferably designed to provide enhanced guidance for caregivers in delivering oral solutions to children. However, such methods can be adapted to dosing in adults as well. Medicines are known to comprise many types of active ingredients including, but not limited to: (1) analgesics, illustrated by, albeit not limited to aspirin, acetaminophen, and/or ibuprofen; (2) antihistamines illustrated by, albeit not limited to brompheniramine maleate, chlorcyclizine hydrochloride, chlorpheniramine maleate, dexbrompheniramine maleate, dexchlorpheniramine maleate, diphenhydramine citrate, diphenhydramine hydrochloride, doxylamine succinate, phenindamine tartrate, pheniramine maleate, pyrilamine maleate, thonzylamine hydrochloride, and/or triprolidine hydrochloride; (3) antitussives illustrated by, albeit not limited to chlophedianol hydrochloride, codeine, codeine phosphate, codeine sulfate, dextromethorphan, dextromethorphan hydrobromide, diphenhydramine citrate, and/or diphenhydramine hydrochloride; (4) bronchodilators illustrated by, albeit not limited to ephedrine, ephedrine hydrochloride, ephedrine sulfate, racephedrine hydrochloride, and/or racepinephrine hydrochloride; (5) expectorants illustrated by, albeit not limited to guaifenesin; and/or (6) nasal decongestants illustrated by, albeit not limited to phenylephrine hydrochloride, pseudoephedrine hydrochloride, pseudoephedrine sulfate, and/or phenylephrine bitartrate in an effervescent dosage form; and (7) antibiotics illustrated by, albeit not limited to penicillins, and/or tetracyclines; and/or their use in combination. The medicines may be safely and optimally dosed by doctor's prescription based on a patient's weight, or other means to estimate their body fluid volume to determine the dosage required to determine the effective and safe units of active ingredients per unit of patient's weight, mass, volume, and/or body surface area.

While traditional prescription drugs have been a main stay for consumers, use of non-prescription drugs and other fluids can benefit from a weight based-dosing scheme. Vital fluids, which may contain one or more of plasmas, water, sugars, salts, minerals, bodily cells, enzymes, and/or hormones, may benefit from weight-based dosing system as the optimal requirements for such fluids may be determined by a patient's weight or other means to estimate their optimal fluid volume typically administered intravenously. Nutritional supplements, known to comprise vitamins, amino acids and/or substances comprising the same, minerals, sugars, and salts, provide consumers alternative or supplement to traditional means of treating disease. Even if the nutritional supplements can not be used to treat or cure a specific disease, consumers still use nutritional supplements to maintain a healthy lifestyle in hopes of preventing diseases. Similar to traditional drugs and vital fluids, nutritional supplements may be dispensed based on optimal requirements determined by the patient's weight or other means to estimate their optimal fluid volume and/or nutritional requirements typically administered via ingestion.

Optimal safe dosing of medicine, vital fluids, and/or nutritional supplements typically requires a dosing precision being no coarser than 2 mL of fluid volume or equivalent, and most ideally less than 1 mL of fluid volume or equivalent. Non-prescription drugs are specified to be dosed with no greater precision than of 2½ mL representing roughly ½ a teaspoon, and typically dosed with an even coarser precision of 5 mL of fluid volume or equivalent, representing roughly 1 teaspoon. While a medical professional might refer to various literature references in order to formulate a particular dosage regimen for an individual, such references are not easily understood by a lay person, and any attempt to interpret weight-based dosing instructions, absent the input of a medical professional, could result in overdosing or underdosing.

Guaifenesin is a common active ingredient sold as an over-the-counter (OTC) medication. It is primarily used to relieve chest congestion, having the chemical name of 3-(2-methoxyphenoxy)-1,2-propanediol and the structure of:

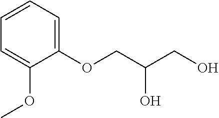

It is generally recognized as safe and effective as an expectorant and works by helping to loosen the phlegm (mucus) and thin bronchial secretions, thereby freeing the bronchial passage of mucus, draining the bronchial passages, and making coughing more productive. It typically exists as the sole active ingredient drug or in combination with antihistamines, cough suppressants, and decongestants. Accordingly, Guaifenesin, individually or in combination, can be delivered to individuals through many forms, including by tablet, capsule, extended-release (long-acting) tablet, dissolving granules, or by solution (syrup or other liquid formulations) taken by mouth. The tablets, capsules, dissolving granules, and syrup are usually taken with or without food every 4 hours as needed. The extended-release tablet is usually taken with or without food every 12 hours. Individuals using this drug are advised to follow the directions on the package or on the prescription label carefully, to ask their doctor or pharmacist to explain any part they do not understand, and to take Guaifenesin exactly as directed.

While Guaifenesin usage is generally seen as safe, proper dosing and its correct use is vital to ensure maximum benefit. Safe administration and usage of most drugs is easier in adults than in children, and users are cautioned that nonprescription cough and cold combination products, including products that contain Guaifenesin, can cause serious side effects or death in young children. Most over-the-counter products containing Guaifenesin contain warnings instructing users not to use the product for children under the age of 4 years old without the consent from their doctor. For children between the ages of 4 and 12 years old, use of such drugs is advisable provided the individuals use caution and follow the package directions carefully. Typically, such individuals are warned that before giving a Guaifenesin product to a child, they must check the package label to find out how much medication the child should receive, and to give the dose that matches the child's age on the chart. Based on age ranges, individuals are instructed to give their children a certain volume, usually measured in teaspoons, every four hours. The problem associated with this dosing scheme is that several age groups are grouped together. For example, it is not uncommon for a recommendation to parents to give 1-2 teaspoons for children ages 6 years old to under 12 years old. For children between the ages of 4 years old and under 6, they are instructed to administer 0.5 to 1 teaspoon. Since these dosage recommendations contain wide age ranges, the appropriate amount of active ingredient may not be delivered in an effective manner, decreasing the likelihood that the drug will perform as needed.

Given the gravity of side effects which might occur from inaccurate dosing, the present invention greatly reduces the possibility of side effects, particularly in young children. Specifically, the present invention teaches a novel guaifenesin oral solution, meant for every 4 hour dosing, having a guaifenesin concentration of, for example, 100 mg/5 mL, which is administered by a novel method of administration in accordance with the following table:

TABLE 1
Dosing scheme based on Active ingredient
per 5 mL: Guaifenesin 100 mg

| | | Minimum | Minimum | Ideal |
|---|---|---|---|---|
| Weight | Ideal Dose | Dose | Guaifenesin | Guaifenesin |
| (lbs) | (mL) | (mL) | Dose (mg) | Dose (mg) |
| 36 | 3.76 | 3.5 | 70 | 75.2 |
| 38 | 3.97 | 3.5 | 70 | 79.4 |
| 40 | 4.17 | 4.0 | 80 | 83.4 |
| 42 | 4.38 | 4.0 | 80 | 87.6 |
| 44 | 4.59 | 4.5 | 90 | 91.8 |
| 46 | 4.79 | 4.5 | 90 | 95.8 |
| 48 | 5 | 5.0 | 100 | 100 |
| 50 | 5.21 | 5.0 | 100 | 104.2 |
| 52 | 5.41 | 5.0 | 100 | 108.2 |
| 54 | 5.62 | 5.5 | 110 | 112.4 |
| 56 | 5.85 | 5.5 | 110 | 117 |
| 58 | 6.06 | 6.0 | 120 | 121.2 |
| 60 | 6.26 | 6.0 | 120 | 125.2 |
| 62 | 6.47 | 6.0 | 120 | 129.4 |
| 64 | 6.67 | 6.5 | 130 | 133.4 |
| 66 | 6.88 | 6.5 | 130 | 137.6 |
| 68 | 7.09 | 7.0 | 140 | 141.8 |
| 70 | 7.29 | 7.0 | 140 | 145.8 |
| 72 | 7.5 | 7.5 | 150 | 150 |
| 74 | 7.71 | 7.5 | 150 | 154.2 |
| 76 | 7.91 | 7.5 | 150 | 158.2 |
| 78 | 8.14 | 8.0 | 160 | 162.8 |
| 80 | 8.35 | 8.0 | 160 | 167 |
| 82 | 8.56 | 8.5 | 170 | 171.2 |
| 84 | 8.76 | 8.5 | 170 | 175.2 |
| 86 | 8.97 | 8.5 | 170 | 179.4 |
| 88 | 9.17 | 9.0 | 180 | 183.4 |
| 90 | 9.38 | 9.0 | 180 | 187.6 |
| 92 | 9.59 | 9.5 | 190 | 191.8 |
| 94 | 9.79 | 9.5 | 190 | 195.8 |
| 95 | 10 | 10.0 | 200 | 200 |

As illustrated by Table 1, the method of dispensing the oral solution having Guaifenesin as the active ingredient, and the oral solution formulated to dispense according to the dosing system, is designed to deliver pediatric dosages for cough and cold products for children less than 12 years of age. The method in accordance with the present invention is designed to improve dosing in pediatric patients by providing a methodology that utilizes the weight of the patient instead of the age. The active ingredient can be dosed so that administration of each dosing unit, i.e. the single dosage in a volume which is taken by the individual, and/or the total daily dosage, would not exceed any current recommended age-based dosing requirements as outlined by FDA monographs for Guaifenesin.

As an illustrative example, the method in accordance with the present invention includes providing an oral solution in the form of an over-the-counter drug having Guaifenesin, preferably having a concentration of 20 mg/mL, to an individual less than 12 years old. The weight of the individual to receive the oral solution is determined. The appropriate amount of volume of the oral solution, which corresponds to the appropriate amount of the active ingredient for the individual's weight, is given to the individual. Referring to Table 1, for an individual that weighs 66 pounds and falls within the age range of 6 years old to 12 years old, the proper amount to be given falls within the range of 6.5 to 6.88 mL, preferably 6.88 mL. Within this range, the individual receives a dose of 130 mg to 137.6 mg of Guaifenesin each time a dose is administered. In contrast to a weight based dosing scheme, the individual following a typical age-based dosing for a Guaifenesin product would be instructed to take 5.0 to 10.0 mL per dose. Within this range, the individual would receive 100 to 200 mg Guaifenesin, representing the possibility of underdosing or overdosing. While overdosing of Guaifenesin is relatively rear because of the clearance of the drug, other active ingredients may cause potential problems. For example, if Guaifenesin is combined with fever and pain reducing ingredients, such as acetaminophen, a delivery scheme that results in overdosing can result in serious complications for the child. Acetaminophen, when taken properly, is safe and effective. The problem with using such a drug, particularly in children who are more susceptible to harm than adults, is administrating the proper dosage. Overdosing of acetaminophen, if not treated immediately, can result in long term liver damage, and if serious enough can result in the liver shutting down, resulting in death to the child. Even if the parent is cautious and under doses the child, the drug may not work as intended, thereby failing to provide relief to the individual. Although underdosing may not directly result in medical complications, such actions may have an indirect effect. As parents realize that their child has not obtained any relief from the first dosage, they may be tempted to administer another dose. This dose may be given earlier than the recommended dosage intervals and the parent may increase the amount given leading to overdosing.

In addition to providing more accurate administration, a method of administrating Guaifenesin based on weight takes into account the needs of children who are in the tenth or ninetieth percentile weight for their particular age group. As described previously, most age based dosing utilizes a large range of ages within a dosing group. Even if the range was limited to one year intervals, say for example, recommending a dosage for each year, i.e. a dosing for 5 year olds, a dosing for 6 year olds, such dosing method does not account for the differences in size that children may have within that year age group. As a result, children within the same age may require different dosing. Children within an age group, particularly young children, may have different builds, resulting in kids who are larger or smaller than what may be determined as the standard. Those children that are smaller or larger, therefore, are at risk of problems associated with underdosing and overdosing. Moreover, children in today's society tend to have a more sedentary lifestyle, resulting in obesity. Obese children, therefore, may not fit into the standard norm for children of a specific age group and would benefit from a method of administering Guaifenesin based on weight.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of administering an oral solution containing one or more active ingredients to an individual less than 12 years old according to weight-based dosing parameters comprising:
providing an oral solution having an effective amount of one or more active ingredients;
determining the weight of an individual less than 12 years old; and
administrating an appropriate amount of said oral solution in a dose dispensing unit in accordance with the weight determination of said individual, whereby the total amount of active ingredient administered to said individual corresponds to the effective amount based on weight.

2. The method of administering an oral solution containing one or more active ingredients to an individual less than 12 years old according to weight-based dosing parameters according to claim 1 wherein said oral solution is a non-prescription medication.

3. The method of administering an oral solution containing one or more active ingredients to an individual less than 12 years old according to weight-based dosing parameters according to claim 1 wherein said oral solution is a vital fluid.

4. The method of administering an oral solution containing one or more active ingredients to an individual less than 12 years old according to weight-based dosing parameters according to claim 1 wherein said oral solution is a nutritional supplement.

5. The method of administering an oral solution containing one or more active ingredients to an individual less than 12 years old according to weight-based dosing parameters according to claim 2 wherein said active ingredient includes guaifenesin.

6. The method of administering an oral solution containing one or more active ingredients to an individual less than 12 years old according to weight-based dosing parameters according to claim 5 wherein said guaifenesin has a concentration of 20 mg/mL.

7. The method of administering an oral solution containing one or more active ingredients to an individual less than 12 years old according to weight-based dosing parameters according to claim 6 wherein said weight determination of 35 to 36 pounds requires administration of between 3.5 mL to 3.76 mL of said oral solution to deliver guaifenesin in a dosage of 70 to 75.1 mg per dosing unit.

8. The method of administering an oral solution containing one or more active ingredients to an individual less than 12 years old according to weight-based dosing parameters according to claim 6 wherein said weight determination of 37 to 38 pounds requires administration of between 3.5 mL to 3.97 mL of said oral solution to deliver guaifenesin in a dosage of 70 to 79.4 mg per dosing unit.

9. The method of administering an oral solution containing one or more active ingredients to an individual less than 12 years old according to weight-based dosing parameters according to claim 6 wherein said weight determination of 39 to 40 pounds requires administration of between 4.0 mL to 4.17 mL of said oral solution to deliver guaifenesin in a dosage of 80 to 84.4 mg per dosing unit.

10. The method of administering an oral solution containing one or more active ingredients to an individual less than 12 years old according to weight-based dosing parameters according to claim 6 wherein said weight determination of 41 to 42 pounds requires administration of between 4.0 mL to 4.38 mL of said oral solution to deliver guaifenesin in a dosage of 80 to 87.6 mg per dosing unit.

11. The method of administering an oral solution containing one or more active ingredients to an individual less than 12 years old according to weight-based dosing parameters according to claim 6 wherein said weight determination of 43 to 44 pounds requires administration of between 4.5 mL to 4.59 mL of said oral solution to deliver guaifenesin in a dosage of 90 to 91.8 mg per dosing unit.

12. The method of administering an oral solution containing one or more active ingredients to an individual less than 12 years old according to weight-based dosing parameters according to claim 6 wherein said weight determination of 45 to 46 pounds requires administration between 4.5 mL to 4.79 mL of said oral solution to deliver guaifenesin in a dosage of 90 to 95.8 mg per dosing unit.

13. The method of administering an oral solution containing one or more active ingredients to an individual less than 12 years old according to weight-based dosing parameters according to claim 6 wherein said weight determination of 47 to 48 pounds requires administration of 5 mL of said oral solution to deliver guaifenesin in a dosage of 100 mg per dosing unit.

14. The method of administering an oral solution containing one or more active ingredients to an individual less than 12 years old according to weight-based dosing parameters according to claim 6 wherein said weight determination of 49 to 50 pounds requires administration of between 5.0 mL to 5.21 mL of said oral solution to deliver guaifenesin in a dosage of 100 to 104.2 mg per dosing unit.

15. The method of administering an oral solution containing one or more active ingredients to an individual less than 12 years old according to weight-based dosing parameters according to claim 6 wherein said weight determination of 51 to 52 pounds requires administration of between 5.0 mL to 5.41 mL of said oral solution to deliver guaifenesin in a dosage of 100 to 108.2 mg per dosing unit.

16. The method of administering an oral solution containing one or more active ingredients to an individual less than 12 years old according to weight-based dosing parameters according to claim 6 wherein said weight determination of 53 to 54 pounds requires administration of a dosing unit of between 5.5 mL to 5.62 mL of said oral solution to deliver guaifenesin in a dosage of 110 to 112.4 mg per dosing unit.

17. The method of administering an oral solution containing one or more active ingredients to an individual less than 12 years old according to weight-based dosing parameters according to claim 6 wherein said weight determination of 55 to 56 pounds requires administration of between 5.5 mL to 5.85 mL of said oral solution to deliver guaifenesin in a dosage of 110 to 117 mg per dosing unit.

18. The method of administering an oral solution containing one or more active ingredients to an individual less than 12 years old according to weight-based dosing parameters according to claim 6 wherein said weight determination of 57 to 58 pounds requires administration of between 6.0 mL to 6.06 mL of said oral solution to deliver guaifenesin in a dosage of 120 to 121.2 mg per dosing unit.

19. The method of administering an oral solution containing one or more active ingredients to an individual less than 12 years old according to weight-based dosing parameters according to claim 6 wherein said weight determination of 59 to 60 pounds requires administration of between 6.0 mL to 6.26 mL of said oral solution to deliver guaifenesin in a dosage of 120 to 125.2 mg per dosing unit.

20. The method of administering an oral solution containing one or more active ingredients to an individual less than 12 years old according to weight-based dosing parameters according to claim 6 wherein said weight determination of 61 to 62 pounds requires administration of between 6.0 mL to 6.47 mL of said oral solution to deliver guaifenesin in a dosage of 120 to 129.4 mg per dosing unit.

21. The method of administering an oral solution containing one or more active ingredients to individual less than 12 years old according to weight-based dosing parameters according to claim 6 wherein said weight determination of 63 to 64 pounds requires administration of between 6.5 mL to 6.67 mL to said oral solution to deliver guaifenesin in a dosage of 130 to 133.4 mg per dosing unit.

22. The method of administering an oral solution containing one or more active ingredients to an individual less than 12 years old according to weight-based dosing parameters according to claim 6 wherein said weight determination of 65 to 66 pounds requires administration of between 6.5 mL to 6.88 mL of said oral solution to deliver guaifenesin in a dosage of 130 to 137.6 mg per dosing unit.

23. The method of administering an oral solution containing one or more active ingredients to an individual less than 12 years old according to weight-based dosing parameters according to claim 6 wherein said weight determination of 67 to 68 pounds requires administration of between 7.0 mL to 7.09 mL of said oral solution to deliver guaifenesin in a dosage of 140 to 141.8 mg per dosing unit.

24. The method of administering an oral solution containing one or more active ingredients to an individual less than 12 years old according to weight-based dosing parameters according to claim 6 wherein said weight determination of 69 to 70 pounds requires administration of between 7.0 mL to 7.29 mL of said oral solution to deliver guaifenesin in a dosage of 140 to 145.8 mg per dosing unit.

25. The method of administering an oral solution containing one or more active ingredients to an individual less than 12 years old according to weight-based dosing parameters according to claim 6 wherein said weight determination of 71 to 72 pounds requires administration of 7.5 mL of said oral solution to deliver guaifenesin in a dosage of 150 mg per dosing unit.

26. The method of administering an oral solution containing one or more active ingredients to an individual less than 12 years old according to weight-based dosing parameters according to claim 6 wherein said weight determination of 73 to 74 pounds requires administration of 7.5 mL to 7.71 mL of said oral solution to deliver guaifenesin in a dosage of 150 to 154.2 mg per dosing unit.

27. The method of administering an oral solution containing one or more active ingredients to an individual less than 12 years old according to weight-based dosing parameters according to claim 6 wherein said weight determination of 75 to 76 pounds requires administration of 7.5 mL to 7.91 mL of said oral solution to deliver guaifenesin in a dosage of 150 to 158.2 mg per dosing unit.

28. The method of administering an oral solution containing one or more active ingredients to an individual less than 12 years old according to weight-based dosing parameters according to claim 6 wherein said weight determination of 77 to 78 pounds requires administration of between 8.0 mL to 8.14 mL of said oral solution to deliver guaifenesin in a dosage of 160 to 162.8 mg per dosing unit.

29. The method of administering an oral solution containing one or more active ingredients to an individual less than 12 years old according to weight-based dosing parameters according to claim 6 wherein said weight determination of 79 to 80 pounds requires administration of between 8.0 mL to 8.5 mL of said oral solution to deliver guaifenesin in a dosage of 70 to 75.1 mg per dosing unit.

30. The method of administering an oral solution containing one or more active ingredients to an individual less than 12 years old according to weight-based dosing parameters according to claim 6 wherein said weight determination of 81 to 82 pounds requires administration of between 8.5 mL to 8.56 mL of said oral solution to deliver guaifenesin in a dosage of 170 to 171.2 mg per dosing unit.

31. The method of administering an oral solution containing one or more active ingredients to an individual less than 12 years old according to weight-based dosing parameters according to claim 6 wherein said weight determination of 83 to 84 pounds requires administration of between 8.5 mL to 8.56 mL of said oral solution to deliver guaifenesin in a dosage of 170 to 175.2 mg per dosing unit.

32. The method of administering an oral solution containing one or more active ingredients to an individual less than 12 years old according to weight-based dosing parameters according to claim 6 wherein said weight determination of 85 to 86 pounds requires administration of between 8.5 mL to 8.97 mL of said oral solution to deliver guaifenesin in a dosage of 170 to 179.4 mg per dosing unit.

33. The method of administering an oral solution containing one or more active ingredients to an individual less than 12 years old according to weight-based dosing parameters according to claim 6 wherein said weight determination of 87 to 88 pounds requires administration of between 9.0 mL to 9.17 mL of said oral solution to deliver guaifenesin in a dosage of 180 to 183.4 mg per dosing unit.

34. The method of administering an oral solution containing one or more active ingredients to an individual less than 12 years old according to weight-based dosing parameters according to claim 6 wherein said weight determination of 89 to 90 pounds requires administration of between 9.0 mL to 9.38 mL of said oral solution to deliver guaifenesin in a dosage of 180 to 187.6 mg per dosing unit.

35. The method of administering an oral solution containing one or more active ingredients to an individual less than 12 years old according to weight-based dosing parameters according to claim 6 wherein said weight determination of 91 to 92 pounds requires administration of between 9.5 mL to 9.59 mL of said oral solution to deliver guaifenesin in a dosage of 190 to 191.8 mg per dosing unit.

36. The method of administering an oral solution containing one or more active ingredients to an individual less than 12 years old according to weight-based dosing parameters according to claim 6 wherein said weight determination of 93 to 94 pounds requires administration of between 9.5 mL to 9.79 mL of said oral solution to deliver guaifenesin in a dosage of 190 to 195.8 mg per dosing unit.

37. The method of administering an oral solution containing one or more active ingredients to an individual less than 12 years old according to weight-based dosing parameters according to claim 6 wherein said weight determination of 95 pounds requires administration of 10 mL of said oral solution to deliver guaifenesin in a dosage of 200 mg per dosing unit.
